(19) United States
(12) Patent Application Publication
(10) Pub. No.: US 2016/0317125 A1
(43) Pub. Date: Nov. 3, 2016

(54) ULTRASONIC DEVICE UNIT, PROBE, ELECTRONIC APPARATUS, AND ULTRASONIC DIAGNOSTIC APPARATUS

(71) Applicant: SEIKO EPSON CORPORATION, Tokyo (JP)

(72) Inventor: Yoshio ARAI, Shiojiri-shi (JP)

(73) Assignee: SEIKO EPSON CORPORATION, Tokyo (JP)

(21) Appl. No.: 15/140,852

(22) Filed: Apr. 28, 2016

(30) Foreign Application Priority Data
Apr. 30, 2015 (JP) 2015-092605

(51) Int. Cl. A61B 8/00; G01S 7/52

(52) U.S. Cl. CPC A61B 8/4483; A61B 8/461; A61B 8/4236; A61B 8/4281; G01S 7/52079

(57) ABSTRACT

An ultrasonic device unit includes ultrasonic transducers disposed in an array. A first multiplexer is connected to the ultrasonic transducers through N element side wiring lines, which are aligned in a first direction along the contour of an element array in plan view, and connects M (M<N) circuit side wiring lines selectively to the element side wiring lines. A second multiplexer is connected to the ultrasonic transducers through L element side wiring lines, which are aligned in a second direction along the contour of the element array in plan view, and connects K (K<L) circuit side wiring lines selectively to the element side wiring lines.

### BACKGROUND

1. Technical Field

The present invention relates to an ultrasonic device unit and a probe, an electronic apparatus, and an ultrasonic diagnostic apparatus using the same.

2. Related Art

As disclosed in JP-A-2012-130564, an ultrasonic device unit having ultrasonic transducer elements arranged in a two-dimensional manner is generally known. In the ultrasonic transducer elements arranged in a two-dimensional manner, a section of a subject can be made to have a spatial thickness. Focus formation or the deflection of an ultrasonic beam can be controlled.

It is difficult to identify the position of a needle from only one two-dimensional image. If a plurality of sectional images are acquired from different directions, the position of the needle is easily identified. For the acquisition of such sectional images, all elements should be separately driven in the ultrasonic transducer elements arranged in a two-dimensional manner. In such separate driving, a signal wiring line is connected to each ultrasonic transducer element. Signal wiring lines should be disposed in a number corresponding to the total number of ultrasonic transducer elements. An increase in the number of wiring lines leads to an increase in the size of the ultrasonic device unit.

### SUMMARY

An advantage of some aspects of the invention is to provide an ultrasonic device unit that can be miniaturized by reducing the number of wiring lines.

(1) An aspect of the invention relates to an ultrasonic device unit including: ultrasonic transducer elements which are disposed in an array and each of which has a vibrating film; a first multiplexer that is connected to the ultrasonic transducer elements through N element side wiring lines, which are aligned in a first direction along a contour of an array in plan view, and connects M (M<N) circuit side wiring lines selectively to the element side wiring lines; and a second multiplexer that is connected to the ultrasonic transducer elements through L element side wiring lines, which are aligned in a second direction crossing the first direction along the contour of the array in plan view, and connects K (K<L) circuit side wiring lines selectively to the element side wiring lines.

In the transmission and reception of ultrasonic waves, an electrical signal is supplied to each ultrasonic transducer element. At this time, in the first multiplexer, at most M circuit side wiring lines are selectively connected to the ultrasonic transducer elements. In the second multiplexer, at most K circuit side wiring lines are selectively connected to the ultrasonic transducer elements. In this manner, the ultrasonic transducer elements are selectively driven. The number of wiring lines drawn from the ultrasonic device unit is reduced from (N+L) to (M+K). The reduction in the number of wiring lines contributes to the miniaturization of the ultrasonic device unit.

(2) The ultrasonic device unit may further include: a first switch that is disposed between the array and the first multiplexer and that is switched between a first position where the element side wiring lines are connected to the array and a second position where the element side wiring lines are disconnected from the array. When a driving signal is supplied to the ultrasonic transducer elements from the first multiplexer, the first switch located at the first position connects the element side wiring lines to the array. The driving signal is supplied to the selected M element side wiring lines. When a driving signal is supplied from the second multiplexer, the first switch located at the second position disconnects the first multiplexer from the array. In this manner, since the circuit side wiring lines of the first multiplexer do not need to be used, control is simplified.

(3) In the ultrasonic device unit, when the first switch is located at the second position, the ultrasonic transducer elements may be grounded. When a driving signal is supplied to one electrode of each ultrasonic transducer element from the second multiplexer, the other electrode of the ultrasonic transducer element is grounded. In this manner, in the ultrasonic transducer elements, a ground potential at the other electrode is common.

(4) The ultrasonic device unit may further include: a second switch that is disposed between the array and the second multiplexer and that is switched between the first position where the element side wiring lines are connected to the array and the second position where the element side wiring lines are disconnected from the array. When a driving signal is supplied to the ultrasonic transducer elements from the second multiplexer, the second switch located at the first position connects the element side wiring lines to the array. The driving signal is supplied to the selected K element side wiring lines. When a driving signal is supplied from the first multiplexer, the second switch located at the second position disconnects the second multiplexer from the array. In this manner, since the circuit side wiring lines of the second multiplexer do not need to be used, control is simplified.

(5) In the ultrasonic device unit, when the second switch is located at the second position, the ultrasonic transducer elements may be grounded. When a driving signal is supplied to the other electrode of each ultrasonic transducer element from the first multiplexer, one electrode of the ultrasonic transducer element is grounded. In this manner, in the ultrasonic transducer elements, a ground potential at one electrode is common.

(6) The ultrasonic device unit may be used by being built into a probe. In this case, the probe may include the ultrasonic device unit and a housing that supports the ultrasonic device unit.

(7) The ultrasonic device unit may be used by being built into an electronic apparatus. In this case, the electronic apparatus may include the ultrasonic device unit and a processing circuit that is connected to the ultrasonic device unit and processes outputs of the ultrasonic transducer elements.

(8) The ultrasonic device unit may be used by being built into an ultrasonic diagnostic apparatus. In this case, the ultrasonic diagnostic apparatus may include the ultrasonic device unit, a processing circuit that is connected to the ultrasonic device unit and processes outputs of the ultrasonic transducer elements to generate an image; and a display device that displays the image.

(9) The ultrasonic device unit may be used by being built into an electronic apparatus. In this case, the electronic apparatus may include the ultrasonic device unit and a control unit that establishes a first mode to set a scanning direction in the second direction in the ultrasonic transducer elements connected to the M element side wiring lines. In the first mode, a sectional image is formed based on the reception signals of the ultrasonic transducer elements arranged along the second direction.

(10) The control unit may establish a second mode to set a scanning direction in the first direction in the ultrasonic transducer elements connected to the K element side wiring lines. In the second mode, a sectional image is formed based on the reception signals of the ultrasonic transducer elements arranged along the first direction.

(11) The control unit may perform switching between the first and second modes. Thus, based on the sections crossing each other, it is possible to acquire an image of an object.

(12) The control unit may include: a line selection section that generates a signal for selecting the element side wiring lines in the first multiplexer; a driving section that generates a driving signal for performing a sector scan of ultrasonic waves, which are transmitted from the ultrasonic transducer elements connected to the selected element side wiring lines, in the second direction; and a detection section that specifies a first direction position and an angular position of the sector scan when a signal strength of a metal body is detected. The ultrasonic device unit can be used in the insertion of a metal body, such as a needle. In general, since the reflectance of the metal body is high, high signal strength is obtained if the ultrasonic wave is perpendicular to the surface of the metal body. Thus, a metal body is specified according to the sector scan.

(13) The line selection section may shift the element side wiring lines in the second direction. If the signal strength of the metal body is eliminated as a result of the shift, the tip (end portion) of the metal body is specified along the first direction. By repeating the shift as described above, the tip of the metal body can be tracked even if the metal body is moved.

(14) The line selection section may select the element side wiring lines in the second multiplexer based on a second direction position. In the first mode, the position of the metal body is specified in the second direction. According to the specified position, in the second mode, the element side wiring lines of the second multiplexer are selected. Therefore, even if the number of element side wiring lines is reduced from L to K in the second multiplexer, the metal body can be reliably tracked in the second mode.

### BRIEF DESCRIPTION OF THE DRAWINGS

The invention will be described with reference to the accompanying drawings, wherein like numbers reference like elements.

FIG. 1 is a schematic diagram that schematically shows the configuration of an ultrasonic diagnostic apparatus.

FIG. 2 is a perspective view schematically showing the surface of an ultrasonic probe.

FIG. 3 is an enlarged partial plan view schematically showing the configuration of an ultrasonic device according to an embodiment.

FIG. 4 is a partial plan view showing the structure of the ultrasonic device in detail.

FIG. 5 is a partial sectional view taken along the line A-A of FIG. 3.

FIG. 6 is a block diagram schematically showing the circuit configuration of the ultrasonic diagnostic apparatus.

FIG. 7 is a conceptual diagram schematically showing an operation of specifying the position of the blood vessel.

FIG. 8 is a conceptual diagram schematically showing an operation of specifying the entire image of a needle.

FIG. 9 is a diagram showing the concept of an image in a long axis mode and an image in a short axis mode.

FIG. 10 is a conceptual diagram showing an operation of specifying a second direction position in detecting the tip of the needle.

### DESCRIPTION OF EXEMPLARY EMBODIMENTS

Hereinafter, an embodiment of the invention will be described with reference to the accompanying diagrams. In addition, the present embodiment to be described below does not unduly limit the content of the invention as defined in the appended claims, and all elements described in the present embodiment are not necessarily indispensable as solving means of the invention.

### (1) Overall Configuration of an Ultrasonic Diagnostic Apparatus

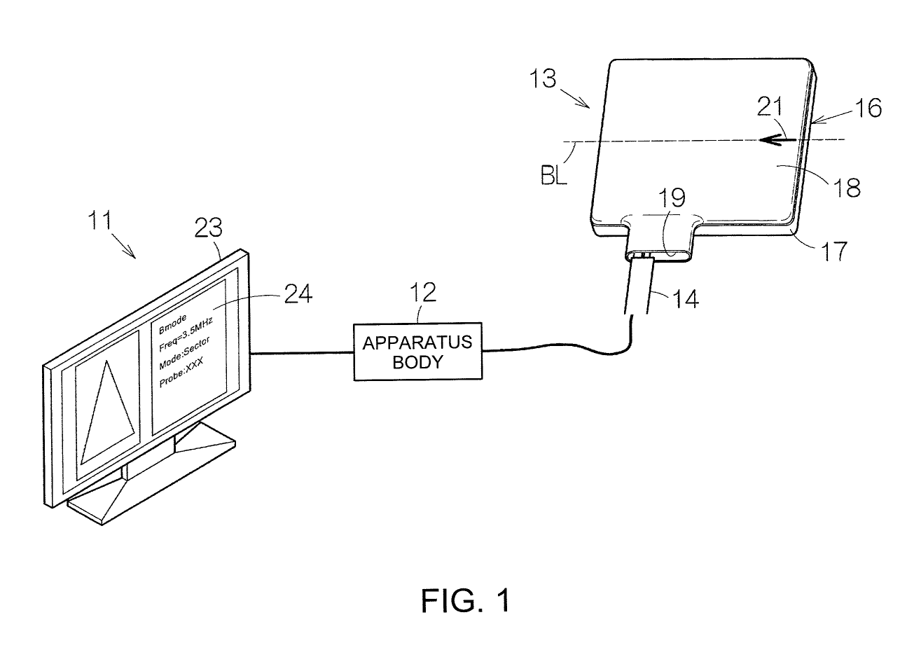

FIG. 1 schematically shows the configuration of a specific example of an electronic apparatus according to an embodiment of the invention, that is, the configuration of an ultrasonic diagnostic apparatus (ultrasonic imaging apparatus) 11. The ultrasonic diagnostic apparatus 11 includes an apparatus body 12 and an ultrasonic probe (probe) 13. The apparatus body 12 and the ultrasonic probe 13 are connected to each other by a cable 14. The apparatus body 12 and the ultrasonic probe 13 perform transmission and reception of electrical signals therebetween through the cable 14.

The ultrasonic probe 13 includes a housing 16. The housing 16 includes a front side body 17 and a back side body 18. The front side body 17 and the back side body 18 are coupled to each other. Between the front side body 17 and the back side body 18, a cable port 19 is provided between the coupling surface of the front side body 17 and the coupling surface of the back side body 18. The cable 14 is disposed in the cable port 19. As will be described later, an ultrasonic device unit is supported by the housing 16.

An insertion mark (marking) 21 is disposed in the housing 16 of the ultrasonic probe 13. The insertion mark 21 is printed, for example, on the surface of the back side body 18. The insertion mark 21 shows the insertion direction of a metal body, such as a needle, along a reference line BL. The insertion mark 21 guides an insertion position for the practitioner of insertion.

A display device 23 is connected to the apparatus body 12. A display panel 24 is built into the display device 23. As will be described later, an image based on the ultrasonic wave detected by the ultrasonic probe 13 is displayed on the screen of the display panel 24. An imaged detection result is displayed on the screen of the display panel 24.

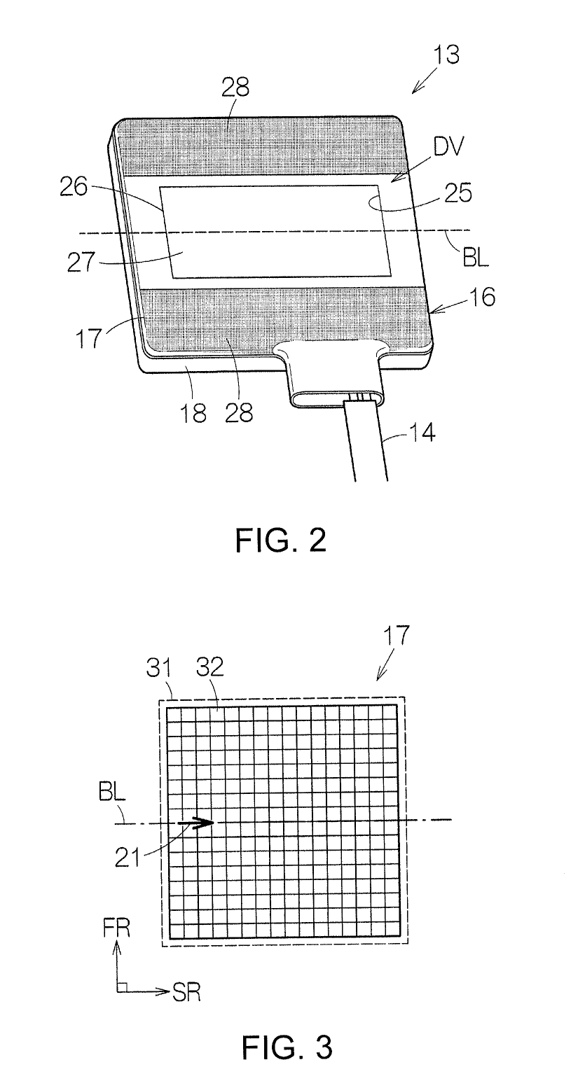

As shown in FIG. 2, an opening 25 is formed in the front side body 17 of the housing 16. The opening 25 faces the housing space provided in the housing 16. An ultrasonic device unit DV is disposed in the housing space. The ultrasonic device unit DV includes an ultrasonic device 26. The ultrasonic device 26 includes an acoustic matching layer 27. The acoustic matching layer 27 is formed of, for example, silicone resin. The acoustic matching layer 27 has an acoustic impedance (for example, 1.0 [MRayl] to 1.5 [MRayl]) close to the acoustic impedance 1.5 [MRayl] of the body. The ultrasonic device 26 outputs an ultrasonic wave from the surface and receives a reflected wave of the ultrasonic wave. The ultrasonic diagnostic apparatus 11 or the ultrasonic probe 13 may have other structures.

The ultrasonic probe 13 has an adhesive layer 28. The adhesive layer 28 is laminated on the surface of the front side body 17, for example. The adhesive layer 28 has adhesion to an object, such as the skin. The ultrasonic probe 13 can be attached to the object due to the adhesive layer 28. If the ultrasonic probe 13 is attached in this manner, the acoustic matching layer 27 is in close contact with the object. In addition, when bringing the acoustic matching layer 27 into close contact with the object, a gel or a thin gel sheet for ultrasonic waves may be interposed between the acoustic matching layer 27 and the object.

### (2) Configuration of the Ultrasonic Device

FIG. 3 schematically shows the configuration of the ultrasonic device 26 according to an embodiment. The ultrasonic device 26 includes an element array (transducer element group) 31. The element array 31 includes ultrasonic transducers 32 that are disposed in an array. In FIG. 3, the ultrasonic transducer 32 is expressed per square mass. Here, the ultrasonic transducers 32 of N rows and L columns are arranged in the element array 31. That is, ultrasonic transducer columns of N rows are arranged in a first direction (hereinafter, referred to as a “short axis direction”) FR, and ultrasonic transducer columns of L columns are arranged in a second direction (hereinafter, referred to as a “long axis direction”) SR perpendicular (at 90 degrees) to the first direction. As will be described later, one ultrasonic transducer 32 includes one or more ultrasonic transducer elements (hereinafter, referred to as “transducer elements”). Each transducer element receives an ultrasonic wave having a determined frequency, and converts the ultrasonic wave into an electrical signal. Each transducer element can transmit an ultrasonic wave having a frequency determined according to the supply of an electrical signal.

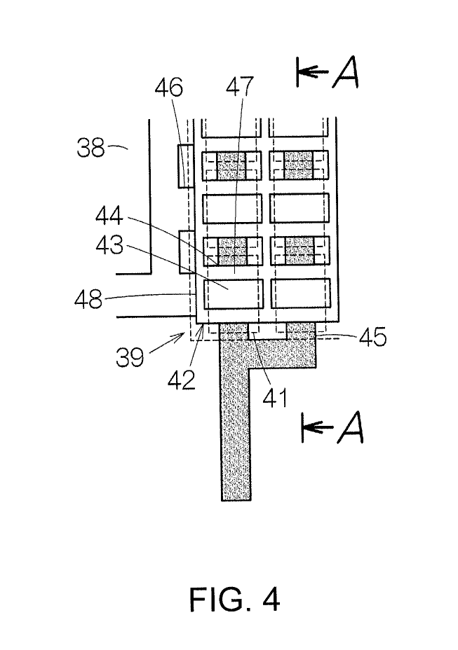

FIG. 4 shows the structure of the ultrasonic device 26 in more detail. The ultrasonic device 26 includes a base 38. A transducer element 39 is formed on the base 38. Each transducer element 39 includes a vibrating film 41. The details of the vibrating film 41 will be described later. In FIG. 4, the contour of the vibrating film 41 is drawn by a dotted line in plan view from a direction perpendicular to the surface of the vibrating film 41 (in plan view from the thickness direction of the substrate). A piezoelectric element 42 is formed on the vibrating film 41. In the piezoelectric element 42, a piezoelectric film (not shown) is interposed between an upper electrode 43 and a lower electrode 44, as will be described later. These are superimposed in order. The ultrasonic device 26 is formed as one ultrasonic transducer element chip.

A plurality of first signal electrode lines 45 are formed on the surface of the base 38. The first signal electrode lines 45 extend in parallel in the column direction of the array. For each ultrasonic transducer 32, the first signal electrode lines 45 are combined into one. The first signal electrode line 45 forms the lower electrode 44 for each transducer element 39. For the first signal electrode line 45, for example, a laminated film of titanium (Ti), iridium (Ir), platinum (Pt), and titanium (Ti) can be used. However, other conductive materials may be used for the first signal electrode line 45.

A plurality of second signal electrode lines 46 are formed on the surface of the base 38. The second signal electrode lines 46 extend in parallel in the row direction of the array. In all of the ultrasonic transducers 32, the second signal electrode lines 46 can be combined into one. The second signal electrode line 46 forms the upper electrode 43 for each transducer element 39. The second signal electrode line 46 can be formed of, for example, iridium (Ir). However, other conductive materials may be used for the second signal electrode line 46.

The supply of power to the transducer element 39 is switched for each ultrasonic transducer 32. Since the transducer elements 39 in each ultrasonic transducer 32 output ultrasonic waves at the same time, the number of transducer elements 39 in each ultrasonic transducer 32 can be determined according to the output level of the ultrasonic wave.

On the vibrating film 41, an electrode separation film 47 is disposed in parallel to the second signal electrode line 46. The electrode separation film 47 extends in a band shape in the longitudinal direction of the second signal electrode line 46. The electrode separation film 47 has an insulation property and a moisture-proof property. The electrode separation film 47 is formed of, for example, a moisture-proof insulating material, such as alumina (Al₂O₃) or silicon oxide (SiO₂). The electrode separation film 47 is formed so as to be separated on both sides of the second signal electrode line 46 with the second signal electrode line 46 interposed therebetween. Since the second signal electrode line 46 crosses the first signal electrode line 45 on the vibrating film 41, the electrode separation film 47 traverses the first signal electrode line 45 on the vibrating film 41.

On the base 38, an insulating film 48 is formed outside the region of the vibrating film 41. The insulating film 48 extends in a band shape in the longitudinal direction of the first signal electrode line 45. The insulating film 48 is disposed in parallel to the first signal electrode line 45 in only a region outside the region of the vibrating film 41. The insulating film 48 is formed of, for example, a moisture-proof insulating material, such as alumina or silicon oxide. The insulating film 48 traverses the second signal electrode line 46. The insulating film 48 is continuous to the electrode separation film 47.

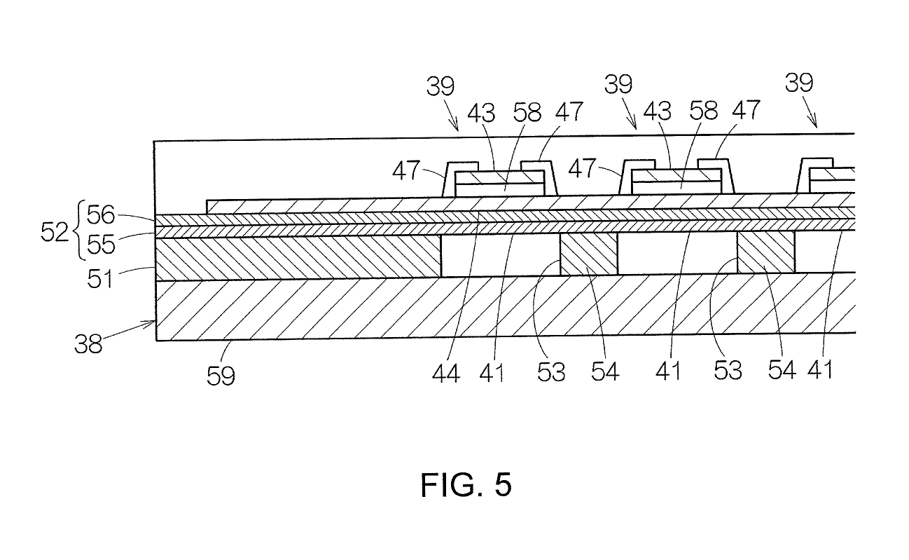

As shown in FIG. 5, the base 38 includes a substrate 51 and a flexible film 52. The flexible film 52 is formed on the entire surface of the substrate 51. On the substrate 51, an opening 53 is formed for each transducer element 39. The openings 53 are disposed in the form of an array for the substrate 51. A partition wall 54 is provided between two adjacent openings 53. The adjacent openings 53 are partitioned by the partition wall 54.

The flexible film 52 is formed by a silicon oxide (SiO₂) layer 55 laminated on the surface of the substrate 51 and a zirconium oxide (ZrO₂) layer 56 laminated on the surface of the silicon oxide layer 55. The flexible film 52 is in contact with each opening 53. In this manner, a part of the flexible film 52 forms the vibrating film 41 corresponding to the contour of the opening 53.

The first signal electrode line 45, a piezoelectric film 58, and the second signal electrode line 46 are laminated in order on the surface of the vibrating film 41. The piezoelectric film 58 can be formed of, for example, lead zirconate titanate (PZT). Other piezoelectric materials may be used for the piezoelectric film 58. Here, the piezoelectric film 58 completely covers the surface of the first signal electrode line 45 under the second signal electrode line 46. A short circuit between the first signal electrode line 45 and the second signal electrode line 46 can be avoided due to the piezoelectric film 58. The surface of the piezoelectric film 58 is covered by the electrode separation film 47.

The acoustic matching layer 27 covers the element array 31. The acoustic matching layer 27 is laminated on the surface of the base 38. A reinforcing plate 59 as a backing material is bonded to the back surface of the base 38. The reinforcing plate 59 is formed in a flat plate shape. The back surface of the base 38 overlaps the surface of the reinforcing plate 59. The surface of the reinforcing plate 59 is bonded to the back surface of the base 38. In such bonding, the reinforcing plate 59 may be bonded to the base 38 with an adhesive. The reinforcing plate 59 increases the rigidity of the base 38. The reinforcing plate 59 can include a rigid base material, for example. Such a base material may be formed of a metal material, such as a 42 alloy (iron-nickel alloy).

### (3) Circuit Configuration of the Ultrasonic Diagnostic Apparatus

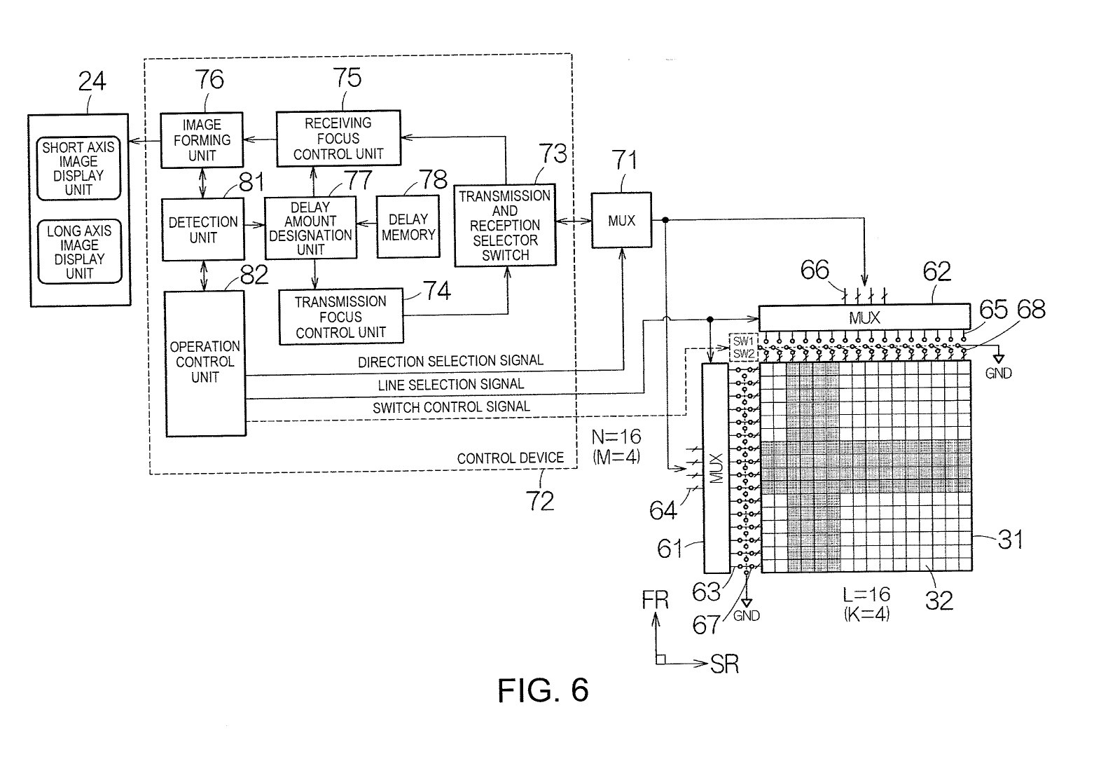

As shown in FIG. 6, a first multiplexer 61 and a second multiplexer 62 are connected to the ultrasonic device 26. The ultrasonic transducers 32 are connected to the first multiplexer 61 through (N×L) element side wiring lines 63 that are aligned in a short axis direction FR along the contour of the element array 31. Similarly, circuit side wiring lines 64 of M (M<N) rows are connected to the first multiplexer 61. The first multiplexer 61 connects the circuit side wiring lines 64 of M rows to the element side wiring lines 63 in response to the supplied line selection signal. Here, since the first signal electrode line 45 is drawn for each ultrasonic transducer 32, L (=16) conductive wires are included in the circuit side wiring lines 64 of one row. A low-noise amplifier may be mounted in the first multiplexer 61.

The ultrasonic transducers 32 are connected to the second multiplexer 62 through (L×N) element side wiring lines 65 that are aligned in the long axis direction SR along the contour of the element array 31. Similarly, circuit side wiring lines 66 of K (<L) columns are connected to the second multiplexer 62. The second multiplexer 62 connects the K circuit side wiring lines 66 to the element side wiring lines 65 in response to the supplied line selection signal. Here, since the second signal electrode line 46 is drawn for each ultrasonic transducer 32, N(=16) conductive wires are included in the circuit side wiring lines 66 of one column. A low-noise amplifier may be mounted in the second multiplexer 62.

A first switch 67 is disposed between the element array 31 and the first multiplexer 61. The first switch 67 is switched between a first position to connect the element side wiring lines 63 to the first signal electrode line 45 of the element array 31 and a second position to disconnect the element side wiring lines 63 from the first signal electrode line 45. In such switching, a switch control signal is supplied to the first switch 67. Here, when the first switch 67 is located at the second position, the ultrasonic transducer 32 is grounded.

A second switch 68 is disposed between the element array 31 and the second multiplexer 62. The second switch 68 is switched between a first position to connect the element side wiring lines 65 to the second signal electrode line 46 of the element array 31 and a second position to disconnect the element side wiring lines 65 from the second signal electrode line 46. In such switching, a switch control signal is supplied to the second switch 68. Here, when the second switch 68 is located at the second position, the ultrasonic transducer 32 is grounded.

The circuit side wiring lines 64 of the first multiplexer 61 and the circuit side wiring lines 66 of the second multiplexer 62 are connected to a third multiplexer 71. The third multiplexer 71 selects the first multiplexer 61 or the second multiplexer 62. In such selection, a direction selection signal is supplied to the third multiplexer 71. For example, the third multiplexer 71 may select the first multiplexer 61 and the second multiplexer 62 alternately. A control device (processing circuit) 72 is connected to the third multiplexer 71. In such connection, M or K (larger one) wiring lines are formed. A control device 72 is built into the apparatus body 12, for example. Wiring lines are housed in a cable 14. In addition to the first multiplexer 61 and the second multiplexer 62, the third multiplexer 71 may be included in the ultrasonic device unit DV.

The display panel 24 is connected to the control device 72. A video signal is supplied to the display panel 24 from the control device 72. Based on the supplied video signal, an ultrasonic image or other pieces of information are displayed on the screen of the display panel 24.

The control device 72 includes a transmission and reception selector switch 73. The transmission and reception selector switch 73 is connected to the third multiplexer 71. A transmission focus control unit (driving unit) 74 and the receiving focus control unit 75 are connected to the transmission and reception selector switch 73. At the time of transmission of ultrasonic waves, the transmission and reception selector switch 73 connects the transmission focus control unit 74 to the third multiplexer 71. At this time, the receiving focus control unit 75 is disconnected from the transmission focus control unit 74 and the third multiplexer 71. At the time of reception of ultrasonic waves, the transmission and reception selector switch 73 connects the receiving focus control unit 75 to the third multiplexer 71. At this time, the transmission focus control unit 74 is disconnected from the receiving focus control unit 75 and the third multiplexer 71. For example, the transmission and reception selector switch 73 may have a clamping function so that a signal having a predetermined voltage or more is not input to the receiving focus control unit 75 at the time of transmission.

The transmission focus control unit 74 outputs a transmission signal to the transducer element 39 through the transmission and reception selector switch 73 at the time of transmission. The transmission focus control unit 74 can include a pulse generator and a delay circuit, for example. The pulse generator outputs a pulse voltage. The pulse voltage is applied to the piezoelectric film 58 of the transducer element 39. In response to the supply of the pulse voltage, the vibrating film 41 performs ultrasonic vibration. In this manner, the ultrasonic wave is transmitted from the transducer element 39. The delay circuit can give a time difference to the application of a voltage to the first signal electrode line 45 or the second signal electrode line 46. The application time difference determines a propagation direction of the ultrasonic wave output from the ultrasonic transducer 32. It is possible to control the transmission direction of the plane wave by changing the delay time.

The receiving focus control unit 75 receives a reception signal of the transducer element 39. An ultrasonic wave reflected from the measurement target causes the vibrating film 41 of the transducer element 39 to perform ultrasonic vibration. In response to the ultrasonic vibration, a reception signal is output from the piezoelectric film 58. The receiving focus control unit 75 can include, for example, a receiving amplifier and a signal processing circuit. A reception signal amplified by the receiving amplifier is subjected to signal processing by a signal processing circuit. The signal processing circuit generates a high-resolution image signal that is focused on a specific object according to aperture combination or adaptive beam forming. In such processing of adaptive beam forming, for example, it is possible to use an MV method or a DCMP method and processing using a covariance matrix, such as MUSIC.

The control device 72 includes an image forming unit 76. The image forming unit 76 is connected to the receiving focus control unit 75. An image signal is supplied to the image forming unit 76 from the receiving focus control unit 75. The image forming unit 76 generates image data based on the supplied image signal.

The control device 72 includes a delay amount designation unit 77. The delay amount designation unit 77 is connected to the transmission focus control unit 74 and the receiving focus control unit 75. The delay amount designation unit 77 designates a delay amount in the delay processing of the transmission focus control unit 74 or the signal processing of the receiving focus control unit 75. The delay amount may be stored in advance in a delay memory 78, for example.

The control device 72 includes a detection unit 81. The detection unit 81 is connected to the image forming unit 76. When the signal strength of the metal body is detected, the detection unit 81 specifies a position in the long axis direction and the angular position of a sector scan.

The control device 72 includes an operation control unit 82 (control unit) 82. The operation control unit 82 is connected to the first multiplexer 61, the second multiplexer 62, and the third multiplexer 71. The detection unit 81 is connected to the operation control unit 82. The operation control unit 82 generates a direction selection signal, a line selection signal, and a switch control signal based on the output of the detection unit 81.

### (4) Operation of the Ultrasonic Diagnostic Apparatus

Next, the operation of the ultrasonic diagnostic apparatus 11 will be described. In the ultrasonic diagnostic apparatus 11, a long axis mode (first mode) and a short axis mode (second mode) are alternately carried out. In the long axis mode, a scanning direction is set in the long axis direction SR in the ultrasonic transducers 32 of M rows that are connected to the (M×L) element side wiring lines 63. In the selection of the (M×L) element side wiring lines 63, a line selection signal is supplied to the first multiplexer 61. The number of wiring lines M is set according to a factor, such as focus formation or the deflection of an ultrasonic beam. In the short axis mode, a scanning direction is set in the short axis direction FR in the ultrasonic transducers 32 of K columns that are connected to the (K×N) element side wiring lines 65. In the selection of the (K×N) element side wiring lines 63, a line selection signal is supplied to the second multiplexer 62. The number of wiring lines K is set according to a factor, such as focus formation or the deflection of an ultrasonic beam. The number of wiring lines M and the number of wiring lines K may be set equally.

The operation control unit 82 performs switching between the long axis mode and the short axis mode. In such switching, the operation control unit 82 generates a direction selection signal and a switch control signal. Either the first multiplexer 61 or the second multiplexer 62 is connected to the third multiplexer 71 by the direction selection signal. When the first multiplexer 61 is connected to the third multiplexer 71, the first switch 67 is located at the first position. The second switch 68 is located at the second position. In this manner, reception signals are output from the M circuit side wiring lines 64. When the second multiplexer 62 is connected to the third multiplexer 71, the second switch 68 is located at the first position. The first switch 67 is located at the second position. In this manner, reception signals are output from the K circuit side wiring lines 66.

For example, a scene of inserting a needle into the blood vessel is assumed. The ultrasonic probe 13 is attached to the arm of the patient for the assistance of needling. In this case, the practitioner of insertion may superimpose the insertion mark 21 on the expected blood vessel. The long axis of the blood vessel is disposed along the reference line BL.

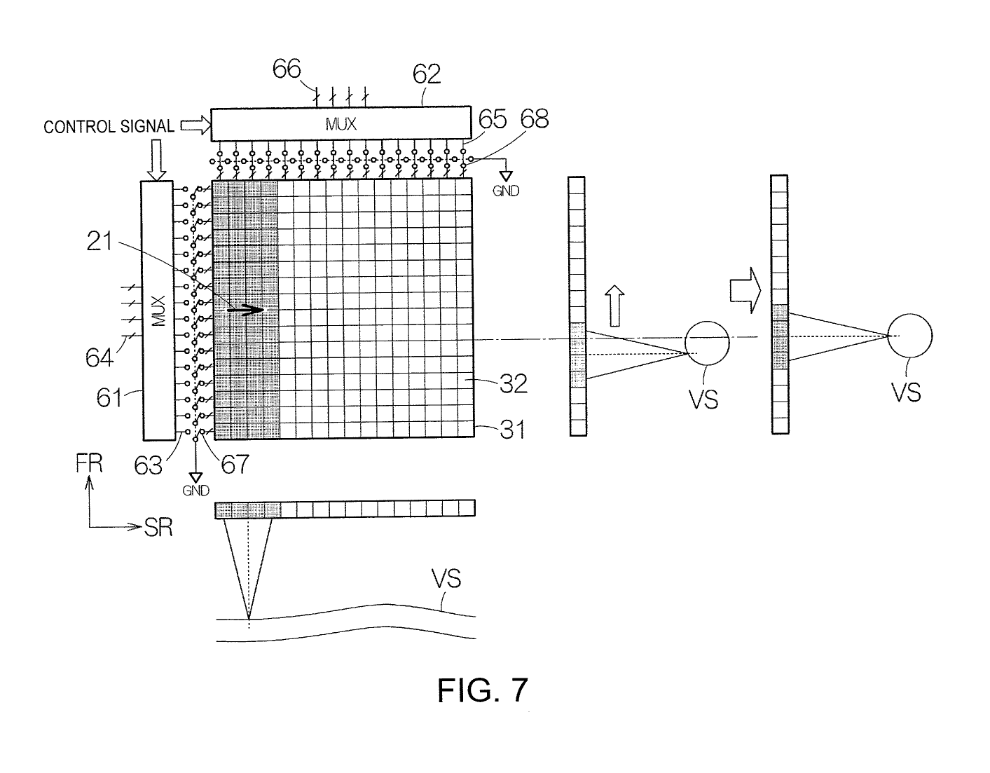

As shown in FIG. 7, prior to the insertion, the position of a blood vessel VS is specified based on the short axis mode. The element side wiring lines 65 of K columns that are selected are shifted in the long axis direction SR. The tomographic image of the blood vessel VS is acquired. As a result, the blood vessel VS is reproduced in a virtual three-dimensional space. On the screen of the display panel 24, for example, an image of the blood vessel VS may be superimposed on an image of the ultrasonic probe 13 in plan view. The practitioner of insertion can see the position of the blood vessel VS relative to the ultrasonic probe 13. The practitioner of insertion can insert a needle toward the specified blood vessel VS.

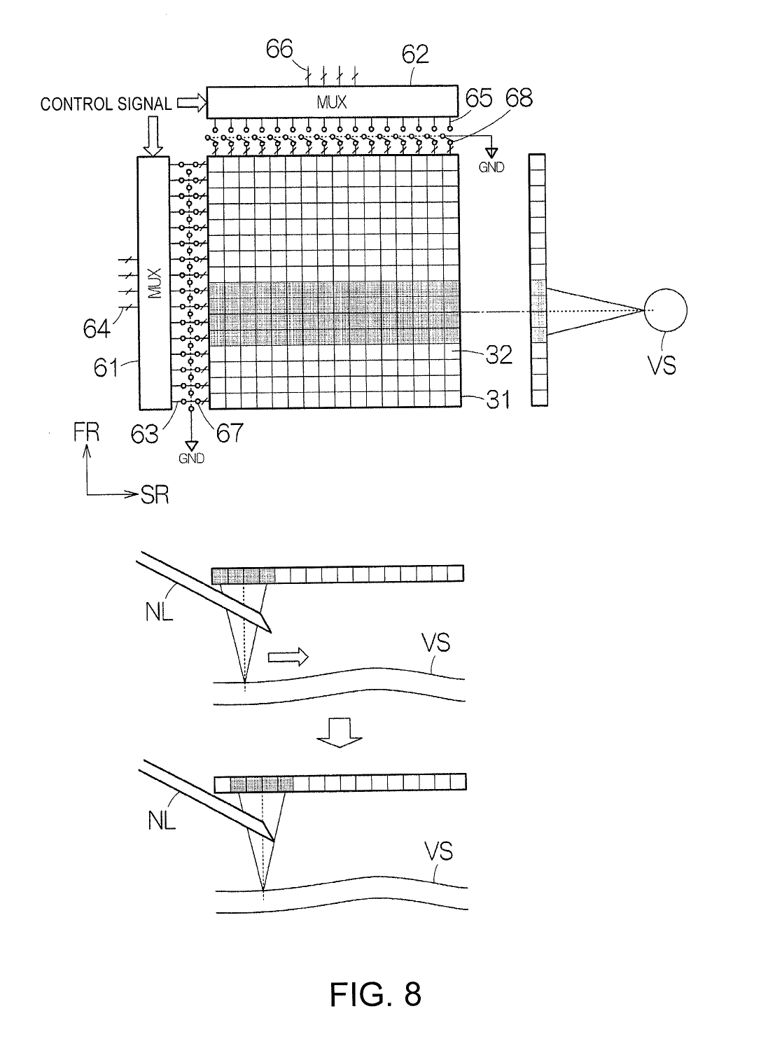

The operation control unit 82 selects the element side wiring lines 63 of M rows selected in the long axis mode according to the direction of the needle. In the long axis mode, an arm is scanned along the shaft center of the needle. As a result, as shown in FIG. 8, the entire image of a needle NL is specified. The tip of the needle NL is specified based on the entire image. Based on such an image, the practitioner of insertion can determine the traveling direction of the needle NL.

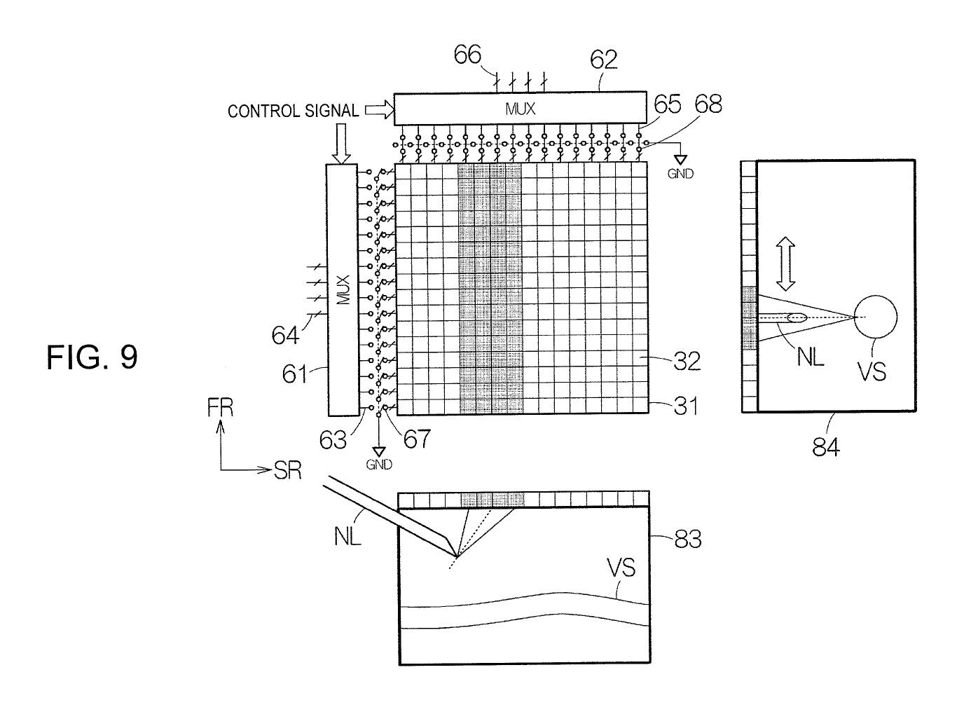

The operation control unit 82 selects the element side wiring lines 65 of K columns that are selected in the short axis mode according to the position of the tip of the needle NL. In the short axis mode, the arm is scanned so as to traverse the axis of the needle NL. As a result, as shown in FIG. 9, the relative positions of the needle NL and the blood vessel VS are specified at the tip of the needle NL. Since an image 83 in the long axis mode and an image 84 in the short axis mode are combined, the practitioner of insertion can make the needle NL proceed toward the blood vessel VS accurately.

In the first multiplexer 61, the circuit side wiring lines 64 of at most M rows are selectively connected to the ultrasonic transducer 32. In the second multiplexer 62, the circuit side wiring lines 66 of at most K columns are selectively connected to the ultrasonic transducer 32. In the third multiplexer 71, wiring lines of M rows or K columns (larger one) are connected to the control device 72. In this manner, the ultrasonic transducers 32 are selectively driven. The number of wiring lines drawn from the ultrasonic device unit DV is reduced from (N×L) to (M×L) or (K+N). The reduction in the number of wiring lines contributes to the miniaturization of the ultrasonic device unit DV.

When a driving signal is supplied to the ultrasonic transducer 32 from the first multiplexer 61, the first switch 67 located at the first position connects the element side wiring lines 63 to the element array 31. A driving signal is supplied to the element side wiring lines 63 of M rows that are selected. When a driving signal is supplied from the second multiplexer 62, the first switch 67 located at the second position disconnects the first multiplexer 61 from the element array 31. The circuit side wiring lines 64 of the first multiplexer 61 do not need to be used. When the first switch 67 is located at the second position, the ultrasonic transducer 32 is grounded. When a driving signal is supplied to one electrode of the ultrasonic transducer 32 from the second multiplexer 62, the other electrode of the ultrasonic transducer 32 is grounded. In each ultrasonic transducer 32, a ground potential at the other electrode is common.

When a driving signal is supplied to the ultrasonic transducer 32 from the second multiplexer 62, the second switch 68 located at the first position connects the element side wiring lines 65 to the element array 31. A driving signal is supplied to the element side wiring lines 65 of K columns that are selected. When a driving signal is supplied from the first multiplexer 61, the second switch 68 located at the second position disconnects the second multiplexer 62 from the element array 31. The circuit side wiring lines 66 of the second multiplexer 62 do not need to be used. When the second switch 68 is located at the second position, the ultrasonic transducer 32 is grounded. When a driving signal is supplied to the other electrode of the ultrasonic transducer 32 from the first multiplexer 61, one electrode of the ultrasonic transducer 32 is grounded. In each ultrasonic transducer 32, a ground potential at one electrode is common.

In the long axis mode, a scanning direction is set in the long axis direction SR in the ultrasonic transducers 32 connected to the element side wiring lines 63 of M rows. In the long axis mode, a sectional image is formed based on the reception signals of the ultrasonic transducers 32 arranged along the long axis direction SR. In the short axis mode, a scanning direction is set in the short axis direction FR in the ultrasonic transducers 32 connected to the element side wiring lines 65 of K columns. In the short axis mode, a sectional image is formed based on the reception signals of the ultrasonic transducers 32 arranged along the short axis direction FR. The long axis mode and the short axis mode are switched to each other. Accordingly, it is possible to acquire the image of an object based on the sections crossing each other.

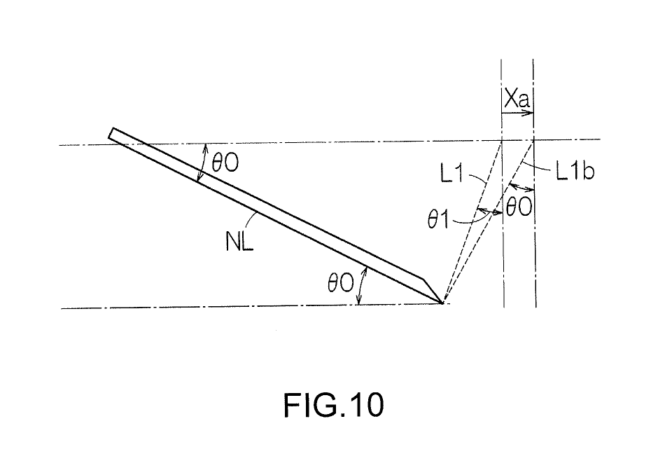

In tracking the needle tip, the element side wiring lines 63 of the first multiplexer 61 are selected in the long axis mode. In such selection, a line selection signal is supplied to the first multiplexer 61 from the operation control unit 82. The transmission focus control unit 74 generates a driving signal for performing a sector scan of ultrasonic waves, which are transmitted from the ultrasonic transducers 32 connected to the selected element side wiring lines 63, in the long axis direction SR. According to the sector scan of ultrasonic waves, the ultrasonic waves are reflected from the needle NL. In response to the reception of ultrasonic waves, a reception signal is output from each ultrasonic transducer 32. The image forming unit 76 specifies the entire image of the needle NL. As shown in FIG. 10, an angular position θ1 from the tip of the needle NL to the element array 31 is detected. The detection unit 81 calculates a second direction position L1b perpendicular to the needle tip from the detected angular position θ1 and a second direction position L1. The operation control unit 82 shifts the element side wiring line 65 to the calculated second direction position L1b. As a result, the ultrasonic wave transmitted from each ultrasonic transducer 32 is perpendicular to the shaft axis of the needle NL at the needle tip of the needle NL. Then, the short axis mode is carried out.

In general, the reflectance of the metal body, such as the needle NL, is high. If the ultrasonic wave is perpendicular to the surface of the metal body, high signal strength is obtained. The needle NL is specified according to the sector scan. In addition, if the angle θ0 at the second direction position L1b is maintained and the ultrasonic transducer 32 is shifted in the second direction, the tip of the needle NL can be specified according to the elimination of the signal strength of the needle NL. By repeating the shift, the tip of the needle NL can be tracked even if the needle NL is moved.

While the present embodiment has been described in detail above, it could be easily understood by those skilled in the art that various changes and modifications thereof could be made without departing from novel matters and effects of the invention. Accordingly, all of such modification examples are included in the range of the invention. For example, in the specification or diagrams, a term which is described at least once together with different terms having a broader meaning or the same meaning can be replaced with the different terms in any parts of the specification or diagrams. In addition, the configurations and operations of the ultrasonic diagnostic apparatus, the ultrasonic probe, the ultrasonic transducer, and the like are not limited to those described in the present embodiment, and various modifications can be made.

The entire disclosure of Japanese Patent Application No. 2015-092605 filed on Apr. 30, 2015 is expressly incorporated by reference herein.

What is claimed is:

1. An ultrasonic device unit, comprising:
ultrasonic transducer elements which are disposed in an array and each of which has a vibrating film;
a first multiplexer that is connected to the ultrasonic transducer elements through N element side wiring lines, which are aligned in a first direction along a contour of an array in plan view, and connects M (M<N) circuit side wiring lines selectively to the element side wiring lines; and
a second multiplexer that is connected to the ultrasonic transducer elements through L element side wiring lines, which are aligned in a second direction crossing the first direction along the contour of the array in plan view, and connects K (K<L) circuit side wiring lines selectively to the element side wiring lines.

2. The ultrasonic device unit according to claim 1, further comprising:
a first switch that is disposed between the array and the first multiplexer and that is switched between a first position where the element side wiring lines are connected to the array and a second position where the element side wiring lines are disconnected from the array.

3. The ultrasonic device unit according to claim 2, wherein, when the first switch is located at the second position, the ultrasonic transducer elements are grounded.

4. The ultrasonic device unit according to claim 2, further comprising:
a second switch that is disposed between the array and the second multiplexer and that is switched between the first position where the element side wiring lines are connected to the array and the second position where the element side wiring lines are disconnected from the array.

5. The ultrasonic device unit according to claim 4,
wherein, when the second switch is located at the second position, the ultrasonic transducer elements are grounded.

6. A probe, comprising:
the ultrasonic device unit according to claim 1; and
a housing that supports the ultrasonic device unit.

7. A probe, comprising:
the ultrasonic device unit according to claim 2; and
a housing that supports the ultrasonic device unit.

8. A probe, comprising:
the ultrasonic device unit according to claim 3; and
a housing that supports the ultrasonic device unit.

9. An electronic apparatus, comprising:
the ultrasonic device unit according to claim 1; and
a processing circuit that is connected to the ultrasonic device unit and processes outputs of the ultrasonic transducer elements.

10. An electronic apparatus, comprising:
the ultrasonic device unit according to claim 2; and
a processing circuit that is connected to the ultrasonic device unit and processes outputs of the ultrasonic transducer elements.

11. An electronic apparatus, comprising:
the ultrasonic device unit according to claim 3; and
a processing circuit that is connected to the ultrasonic device unit and processes outputs of the ultrasonic transducer elements.

12. An ultrasonic diagnostic apparatus, comprising:
the ultrasonic device unit according to claim 1;
a processing circuit that is connected to the ultrasonic device unit and processes outputs of the ultrasonic transducer elements to generate an image; and
a display device that displays the image.

13. An ultrasonic diagnostic apparatus, comprising:
the ultrasonic device unit according to claim 2;
a processing circuit that is connected to the ultrasonic device unit and processes outputs of the ultrasonic transducer elements to generate an image; and
a display device that displays the image.

14. An electronic apparatus, comprising:
the ultrasonic device unit according to claim 4; and
a control unit that establishes a first mode to set a scanning direction in the second direction in the ultrasonic transducer elements connected to the M element side wiring lines.

15. The electronic apparatus according to claim 14,
wherein the control unit establishes a second mode to set a scanning direction in the first direction in the ultrasonic transducer elements connected to the K element side wiring lines.

16. The electronic apparatus according to claim 15,
wherein the control unit performs switching between the first and second modes.

17. The electronic apparatus according to claim 16,
wherein the control unit includes:
a line selection section that generates a signal for selecting the element side wiring lines in the first multiplexer;
a driving section that generates a driving signal for performing a sector scan of ultrasonic waves, which are transmitted from the ultrasonic transducer elements connected to the selected element side wiring lines, in the second direction; and
a detection section that specifies a first direction position and an angular position of the sector scan when a signal strength of a metal body is detected.

18. The electronic apparatus according to claim 17,
wherein the line selection section shifts the element side wiring lines in the second direction.

19. The electronic apparatus according to claim 16,
wherein the line selection section selects the element side wiring lines in the second multiplexer based on a second direction position.
